(19) United States
(12) Patent Application Publication
(10) Pub. No.: US 2024/0308059 A1
(43) Pub. Date: Sep. 19, 2024

(54) LOAD DISTRIBUTION DEVICE FOR IMPROVING THE MOBILITY OF THE CENTER OF MASS OF A USER DURING COMPLEX MOTIONS

(71) Applicant: B-TEMIA INC., St-Augustin-de-Desmaures (CA)

(72) Inventors: Nathaniel ZOSO, St-Augustin-de-Desmaures (CA); Stéphane BÉDARD, St-Augustin-de-Desmaures (CA)

(21) Appl. No.: 18/268,205

(22) PCT Filed: Dec. 18, 2021
(86) PCT No.: PCT/CA2021/051846

§ 371 Date: Jun. 16, 2023

Related U.S. Application Data
(60) Provisional application No. 63/127,806, filed on Dec. 18, 2020.

(51) Int. Cl. B25J 9/00

(52) U.S. Cl. CPC B25J 9/0006

(57) ABSTRACT

A load distribution device for transferring musculoskeletal stress from joints to body segments of the lower extremities of a user. The device includes actuation of the hips and knees that follows and assists the user's movement in a complimentary way. The complimentary assistance and load distribution device combine to reduce the loading on the user's joints and increase the user's strength. By assisting the user's hip and/or knees as needed, the device allows the user to achieve improved strength, reduces the metabolic requirements for motion, and increases comfort during physical activity. The load distribution device follows the user's limbs through the full joint range of motion and can be used in both passive and active modes.

### CROSS-REFERENCE TO RELATED APPLICATIONS

This application claims the benefits of U.S. provisional patent application No. 63/127,806 filed on Dec. 18, 2020, which is herein incorporated by reference.

### TECHNICAL FIELD

The present disclosure relates to a load distribution device for improving the mobility of the center of mass of a user during complex motions.

### BACKGROUND

Lower-body exoskeletons and orthoses provide varying levels of structural and mechanical assistance in specific activities but do so at the cost of reduced joint mobility. Passive devices provide static structural support to the wearer, transferring musculoskeletal stress away from the joints, but lack the ability to provide dynamic assistance. Active solutions provide dynamic assistance in limited situations (e.g., walking gait, sit-to-stand) but do not support complex mobility tasks (e.g., multi-planer motions involving the upper and lower body like swinging a bat, shooting a hockey puck, throwing a ball, or rapid changes in direction and explosive movements of the lower body). Limitations in dynamic assistance devices are due to deficits in their controls (i.e., they are unable to follow the user) or in the range of motion of the supporting structure, limiting the user's ability to optimally control their center of mass in these 3D movements.

Accordingly, there is a need for a device capable of providing dynamic support to transfer musculoskeletal stress away from the wearer's joints without limiting their function in complex mobility movements, in order to improve the mobility of their center of mass in these movements.

### SUMMARY

The present disclosure provides a load distribution device for improving the mobility of the center of mass of a user during complex motions, comprising:
a pelvic support belt configured to be positioned about a lower trunk of the user;
at least one thigh support element including two or more contact areas configured to be positioned in an agonist-antagonist configuration on a posterior part and an anterior part of a thigh of the user, the at least one thigh support element being rotationally connected to the pelvic support belt;
at least one hip joint actuator providing rotational motion of the at least thigh support element with respect to the pelvic support belt;
at least one shank support element including two or more contact areas configured to be positioned in an agonist-antagonist configuration on a posterior part and an anterior part of a shank of the user, the at least one shank support element being rotationally connected to the at least one thigh support element;
at least one knee joint actuator providing rotational motion of the at least one shank support element with respect to the at least one thigh support element;
a plurality of sensors positioned on the pelvic support belt, the at least one thigh support element, the hip joint actuator and the knee joint actuator, and at least one foot sensor configured to be positioned on a foot of the user, the plurality of sensors providing mechanical and biomechanical signals;
a control unit operatively connected to the plurality of sensors and at least one foot sensor for receiving the mechanical and biomechanical signals, the control unit having stored thereon executable instructions for processing and analyzing the mechanical and biomechanical signals and generating motions set-points of movements of the user; and
a power unit operatively connected to the at least one knee joint actuator, the at least one hip joint actuator and the control unit;

wherein the at least one knee joint actuator and the at least one hip joint actuator transfer musculoskeletal stress from joints to body segments of lower extremities of a user, and therefore improve stability of the joints and a range of motion of the body segments, by generating or dissipating biomechanical energy under directions of the control unit according to a computed level of energy corresponding to a musculoskeletal stress reduction at the joints of the lower extremities of the user necessary to compensate movements of the user, the generated or dissipated biomechanical energy being redistributed onto the lower trunk, the thigh and the shank of the user via the pelvic support belt, the at least one thigh support element and the at least one shank support element, respectively.

The present disclosure also provides a load distribution device as above, comprising two thigh support elements, two shank support elements, two hip joint actuators, two knee joint actuators and two feet sensors.

The present disclosure also provides a load distribution device wherein each of the thigh support elements is rotationally connected to an associated shank support element via a knee pivot aligned with a center of rotation of a knee joint of the user, and wherein each of the knee joint actuators are located remotely from the center of rotation of the knee joint of the user, each of the knee joint actuator transmitting rotational motion to a corresponding knee pivot via an extension cable and a flexion cable.

The present disclosure also provides a load distribution device wherein each of the thigh support elements is rotationally connected to the pelvic support belt via a hip pivot aligned with a center of rotation of a hip joint of the user, and wherein each of the hip joint actuators is located remotely from the center of rotation of the hip joint of the user, each of the hip joint actuator transmitting rotational motion to a corresponding hip pivot via an extension cable and a flexion cable.

The present disclosure further provides a load distribution device wherein each of the knee of hip joint actuators may located, for example, medially on a respective side portion of the pelvic support belt, on a lower back portion of the pelvic support belt, on a respective front portion of the thigh of the user, on a respective back portion of the thigh of the user or on a respective portion of the thigh support element between a hip joint of the user and the knee pivot.

The present disclosure further provides a load distribution device further comprising a delocalization mechanism including a deportation structural link having at a first extremity an actuator support element configured to support the knee or hip actuator, and a second extremity having a pivot connection element for connecting to the knee or hip pivot. The actuator support element may be configured to removably support the knee or hip actuator.

The present disclosure also provides an orthotic device comprising:
a proximal support element including at least one contact area configured to be secured to a proximal body portion of a user and distal support element including at least one contact area configured to be secured to a distal body portion of the user, the proximal support element and the distal support element being rotationally connected via a pivot aligned with a center of rotation of a corresponding joint of the user;
at least one actuator rotationally providing rotational motion of the distal support element with respect to the proximal support element, the at least one actuator being located remotely from the center of rotation of the corresponding joint of the user, the actuator transmitting rotational motion to the pivot via an extension cable and a flexion cable.

The prosthetic device may further comprise a delocalization mechanism including a deportation structural link having at a first extremity an actuator support element configured to support the actuator, and a second extremity having a pivot connection element for connecting to the pivot. The actuator support element may be configured to removably support the actuator.

### BRIEF DESCRIPTION OF THE FIGURES

Embodiments of the disclosure will be described by way of examples only with reference to the accompanying drawings, in which:

FIGS. 1A, 1B and 1C are front, side and back views, respectively, of the load distribution device for improving the mobility of the center of mass of a user during complex motions in accordance with an illustrative embodiment of the present disclosure;

FIGS. 2A and 2B are schematic views of the positioning of the knee actuator above the hip actuator in accordance with a first and second alternative embodiments of the present disclosure;

FIG. 3 is a schematic view of the positioning of the knee actuator above the hip actuator and on the lower back of the user in accordance with a third alternative embodiment of the present disclosure;

FIGS. 4A, 4B and 4C are schematic views the positioning of the knee actuator above the hip actuator and showing cable attachments in a shortened state in accordance with a fourth alternative embodiment of the present disclosure;

FIGS. 5A, 5B and 5C are schematic views the positioning of the knee actuator above the hip actuator and showing cable attachments in an extended state in accordance with the fourth alternative embodiment of the present disclosure illustrated in FIGS. 4A, 4B and 4C;

FIG. 6 is a schematic view of the positioning of the knee actuator between the hip actuator and the knee articulation, in accordance with a fifth alternative embodiment of the present disclosure;

FIGS. 7A, 7B, 4C and 7D are schematic views of various cable attachments in accordance with alliterative embodiments of the present disclosure;

FIGS. 8A, 8B and 8C are a perspective elevated view, a side view and a rear view of an actuator delocalization mechanism in accordance with an illustrative embodiment of the present disclosure;

FIG. 9 is a schematic representation of the load distribution device control system in accordance with the illustrative embodiment of the present disclosure;

FIG. 10 is a flow diagram of the load distribution device control process in accordance with a first illustrative embodiment of the present disclosure;

FIG. 11 is a flow diagram of the load distribution device control process in accordance with a second illustrative embodiment wherein the user's hips and knees are assisted;

FIG. 12 is a flow diagram of the load distribution device control process in accordance with a third illustrative embodiment wherein the user's hips and knees are resisted.

Similar references used in different Figures denote similar components.

### DETAILED DESCRIPTION

Generally stated, the non-limitative illustrative embodiment of the present disclosure provides a load distribution device for improving the mobility of the center of mass of a user during complex motions. The function of the load distribution device is to biomechanically support the pelvic structure of a user during complex motions in order to dynamically improve the mobility the user's center of mass in real-time. This enhances the efficiency of the 3D displacements of the user's center of mass, the stability of related joints, and the range of motion of related body segments. Therefore, the load distribution device improves the overall mobility of the user, which results in advantages such as increased ability to perform desired motions (independently of their level of complexity), metabolic gain in motions, and increased sacro-lumbar, hips and knees stability, which in turn may decrease stress in the dorsal and upper body segments of the user. In order to do so, the load distribution device maintains correct alignment with the user's joints throughout their movements, for example during walking, jogging, running, weight-bearing, squatting, jumping, kneeling, using stairs, participation in sporting activities, and in work-related activities. The device includes actuation of the hips and knees that follows and assists the user's movement in a complimentary way. The complimentary assistance and load distribution device combine to reduce the loading on the user's joints and increase the user's strength. By assisting the user's hip and/or knees as needed, the device allows the user to achieve improved strength, reduces the metabolic requirements for motion, and increases comfort during physical activity. The load distribution device follows the user's limbs through the full joint range of motion and can be used in both passive and active modes.

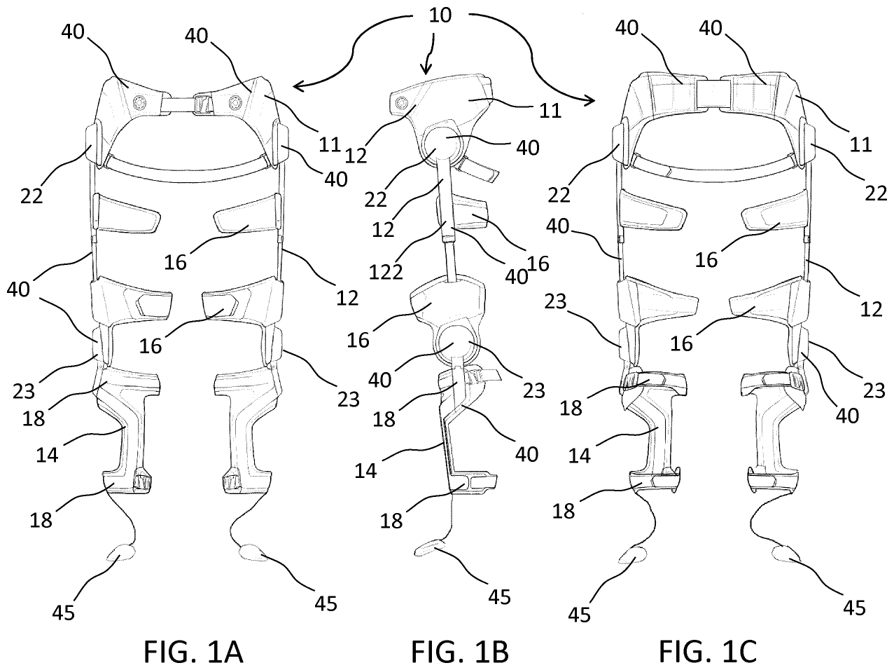

Referring to FIG. 1, the load distribution device for transferring musculoskeletal stress from joints to body segments of the lower extremities of a user 10 comprises a pelvic support belt 11, one or two thigh support elements 12 and one or two shank support elements 14. Advantageously, the pelvic support belt 11 is generally rigid with allowance for some size adjustments, via either extensible portions and/or size adjustment mechanisms.

The pelvic support assembly 11 is configured to be positioned about a lower trunk of the user in an agonist-antagonist configuration and includes a hip joint actuator 22 rotationally connecting the pelvic support belt 11 to the thigh support element 12 and is positioned so as to be aligned with the center of rotation of the hip joint of the user. The hip joint actuator 22 provides active rotational motion at the hip joint of the user. The hip joint actuator 22 may be, for example, an active direct drive rotational actuated mechanism.

The thigh support element 12 include two or more contact areas 16 configured to be positioned in an agonist-antagonist configuration on the posterior and the anterior parts of the thigh of the user.

A knee joint actuator 23 rotationally connect for each of the one or two thigh support elements 12 to the one or two shank support elements 14. The knee joint actuator 23 may be, for example, an active direct drive rotational actuated mechanism.

The shank support element 14 includes two or more contact areas 18 configured to be positioned in an agonist-antagonist configuration on the posterior and the anterior parts of the shank of the user.

A plurality of sensors 40 are positioned on the pelvic support belt 11, either on separate sides or centrally located, the thigh support element 12, the hip joint actuator 22 and the knee joint actuator 23, along with sensors 45 to be positioned at each foot of the user, each of the sensors 40, 45 observing an associated user body segment kinematics in order to provide mechanical and biomechanical information. The sensors 40, 45 may be, for example, inertial and angular sensors.

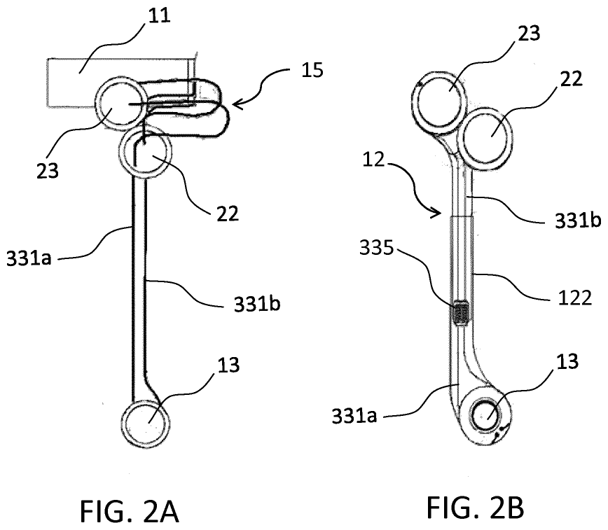

In an alternative embodiment, illustrated in FIG. 2A, the knee joint actuator 23 may be positioned on the pelvic support belt 11 above the hip joint actuator 22, and operatively connected to a knee pivot 13 rotationally connecting the thigh support element 12 to the shank support element 14, in alignment with the center-of-rotation of the knee joint of the user. This allows for a displacement of the weight of the knee joint actuator 23 from the knee joint of the user to the pelvic support belt 11. Rotational motion is transferred from the knee joint actuator 23 to the knee pivot 13 using extension and flexion Bowden cables 331a and 331b, respectively. The Bowden cables 331a, 331b loops 15, which allow for the accommodation of different user heights, are located toward the back of the pelvic support belt 11.

In another alternative embodiment, illustrated in FIG. 2B, the Bowden cables 331a, 331b loop on the knee joint actuator 23. This configuration requires the use of a tensioning mechanism 335 between the knee joint actuator 23 and the knee pivot 13 to manage the tension in the Bowden cables 331a, 331b. This allows for the accommodation of different user heights without having to manage extra cables and avoiding slack so as to obtain good force bandwidth. The tensioning mechanism 335 may be, for example, a Bowden cable tensioner. Screwing the tensioner increases the tension in a corresponding Bowden cable 331a, 331b by elongating its outer casing (sheath).

It should be understood that both the alternative embodiments of FIGS. 2A and 2B are provided with a thigh support element 12 having a length adjustment mechanism 122, for example a slider or a screw mechanism for quick and fine length adjustment.

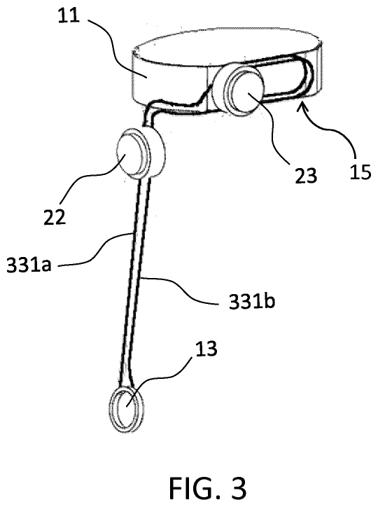

Referring now to FIG. 3, there is shown a further alternative embodiment of the positioning of the knee joint actuator 23, which is positioned on the pelvic support belt 11 above the hip actuator 22 and on the lower back of the user. Power is transferred from the knee joint actuator 23 to the knee pivot 13 using extension and flexion Bowden cables 331a and 331b, respectively. The Bowden cables 331a, 331b loops 15, which allow for the accommodation of different user heights, are located toward the back of the pelvic support belt 11.

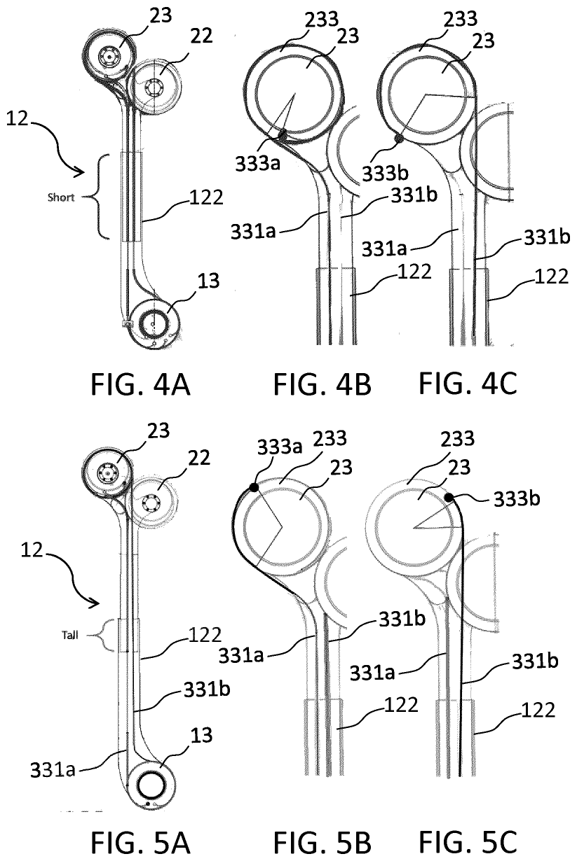

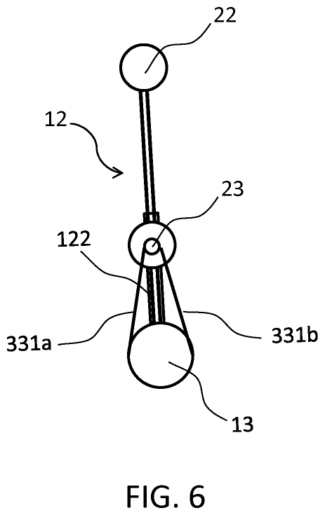

FIGS. 4A, 4B and 4C show a further alternative embodiment of the configuration, in a shortened state, where the knee joint actuator 23 is positioned above the hip actuator 22. In this state, the extension and flexion Bowden cables 331a and 331b are connected to the extension 333a and the flexion 333b proximal cable attachments which are positioned around the knee joint actuator 23 such that the extension and flexion Bowden cables 331a and 331b loop around a major portion of the knee joint actuator 23.

FIGS. 5A, 5B and 5C show the alternative embodiment illustrated in FIGS. 4A, 4B and 4C, in an extended state. In this state, the extension and flexion Bowden cables 331a and 331b are connected to the extension 333a and the flexion 333b proximal cable attachments which are positioned around the knee joint actuator 23 such that the extension and flexion Bowden cables 331a and 331b loop around a minor portion of the knee joint actuator 23.

The positioning of the extension 333a and the flexion 333b proximal cable attachments may be varied, for example, using adjustment pulleys 233 in order to accommodate various thigh support lengths provided by the length adjustment mechanism 122.

FIG. 5 shows a further still alternative embodiment of the positioning of the knee joint actuator 23, which is positioned between the hip joint actuator 22 and the knee pivot 13.

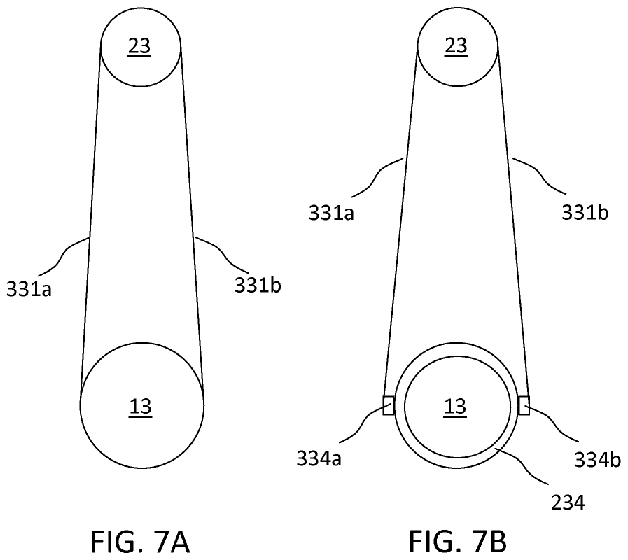

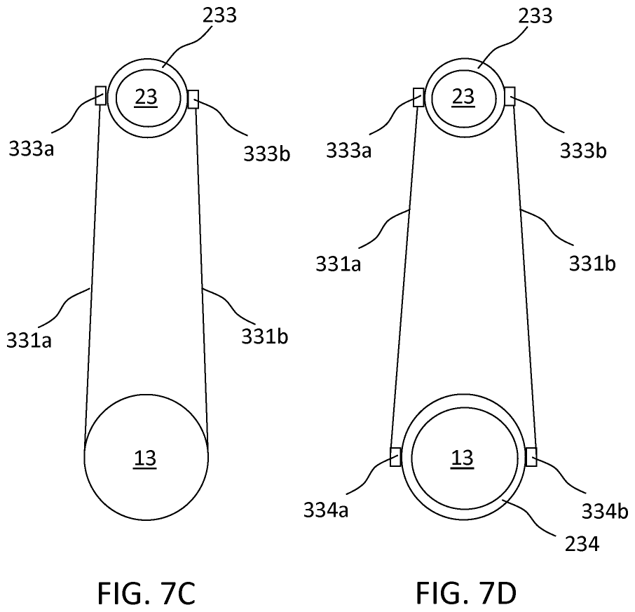

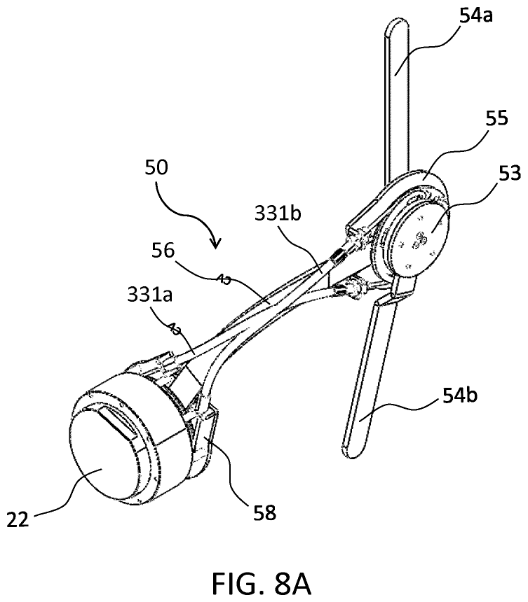

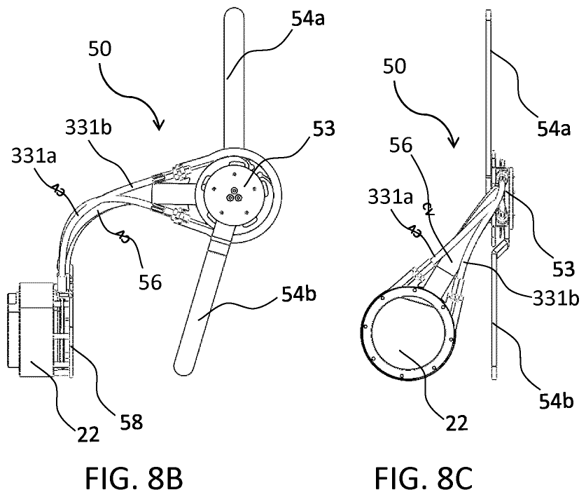

Referring now to FIGS. 7A, 7B, 7C and 7D, there are shown alternative embodiments of the extension 331a and flexion 331b Bowden cables configurations.

In the embodiment of FIG. 7A, the extension 331a and flexion 331b Bowden cables are actually part of a single continuous loop in frictional contact with the stator of the knee joint actuator 23 and knee pivot 13.

In the embodiment of FIG. 7B, the extension 331a and flexion 331b Bowden cables are part of a single cable in frictional contact with the stator of the knee joint actuator 23 and whose two ends are attached to respective extension 334a and flexion 334b distal cable attachments on the knee pivot 13.

In the embodiment of FIG. 7C, the extension 331a and flexion 331b Bowden cables are part of a single cable in frictional contact with the knee pivot 13 and whose two ends are attached to respective extension 333a and flexion 334b proximal cable attachments on the knee joint actuator 23.

In the embodiment of FIG. 7D, the extension 331a and flexion 331b Bowden cables are two separate cables whose ends are attached to respective extension 333a and flexion 333b proximal cable attachments on the knee joint actuator 23 as well as corresponding extension 334a and flexion 334b distal cable attachments on the knee pivot 13.

It is to be understood that the positioning of the proximal 333a, 333b and distal 334a, 334b cable attachments may be varied, for example, using adjustment pulleys 233, 234.

It is to be further understood that in a further alternative embodiment, the hip actuator 22 may be displaced and the hip joint provided with a hip pivot similar to the knee pivot 13, with similar alternative embodiments of placement of the hip actuator as well as extension 331a and flexion 331b Bowden cables configurations, but for the hip actuator and joint instead of the knee actuator and joint.

In another alternative embodiment illustrated in FIGS. º8A, 8B and 8C, the hip joint actuator 22 may be positioned in a delocalized location with respect to the hip joint of the user using a delocalization mechanism 50. The delocalization mechanism 50 operatively connects the hip joint actuator 22 to a hip pivot 53 in alignment with the center-of-rotation of the hip joint of the user, via a deportation structural link 56. The deportation structural link 56 is provided at one extremity with an actuator support element 58, for supporting the hip joint actuator 22, and at another extremity a hip pivot connection element 55, for connecting to the hip pivot 53. The hip pivot 53 rotationally connects the pelvic support belt 11 to the thigh support element 12 via respective fixation segments 54a and 54b. It is to be understood that in an alternative embodiment the hip joint actuator 22 may be removably secured to the actuator support element 58 for easy removal and replacement of the hip joint actuator 22.

The actuator delocalization mechanism 50 is used to move the center of mass of, or simply displace the volume taken by, the hip joint actuator 22 to a more appropriate location depending on the required use of the load distribution device 10. This allows for a displacement of the weight and volume of the hip joint actuator 22 from the hip joint of the user to another position, for example the front or back of the thigh, or the pelvic support belt 11. Rotational motion is transferred from the hip joint actuator 22 to the hip pivot 53 using extension and flexion Bowden cables 331a and 331b, respectively.

Although disclosed with regard to the hip joint actuator 22, it is to be understood that the delocalization mechanism 50 provides the ability to easily delocalize any actuator, for example the knee joint actuator 23, from the targeted joint while keeping the power directly aligned with the joint, which can be used to improve the aesthetics of the load distribution device 10 or other orthotic device, enhance it functionality and/or adjust its effect on the metabolic cost to a user. In another embodiment, the delocalization mechanism 50 may be used to delocalize an ankle actuator, an elbow actuator or a shoulder actuator.

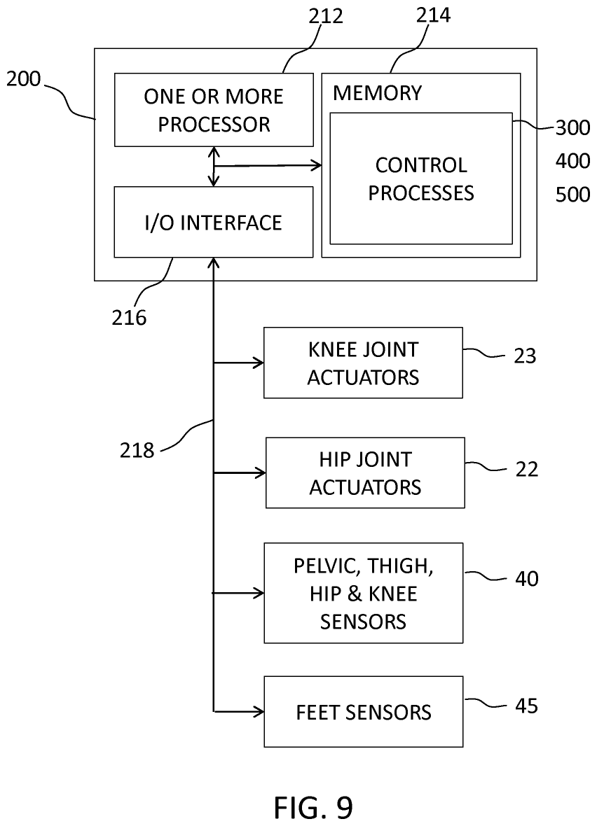

Referring to FIG. 9, control unit 200, which includes load distribution device control processes 300, 400, 500, analyses the mechanical and biomechanical information from the plurality of sensors 40, 45 and provides adaptive tracking and assistance to the user through the load distribution device 10. The load distribution device control system 200 includes one or more processor 212 with an associated memory 214 comprising instructions stored thereon, that when executed on the one or more processor 212, performs the steps of either of the load distribution device control processes 300, 400, 500, which processes will be further described below, and an input/output (I/O) interface 216 for communication with the knee joint actuators 23, the hip joint actuators 22, the pelvic, thigh, hip and knee sensors 40, and the foot sensors 45, through communication link 218, which may be wired, wireless or a combination of both.

A power unit (not shown) provides power to the knee joint actuator 23, the hip joint actuator 22 and the control unit 200.

In use, the knee joint actuator 23 and the hip joint actuator 22 generate and/or dissipate biomechanical energy under directions of the control unit 200 in accordance with user customization and/or mode of operation (e.g., tracking, exercising, etc.), to a computed level of energy corresponding to a musculoskeletal stress reduction, at the joints of the lower extremities of the user, necessary to compensate movements of the user. The generated or dissipated biomechanical energy is then redistributed onto the lower trunk, the thigh and the shank of the user via the pelvic support belt 11, the thigh support element 12 and the shank support element 14, respectively.

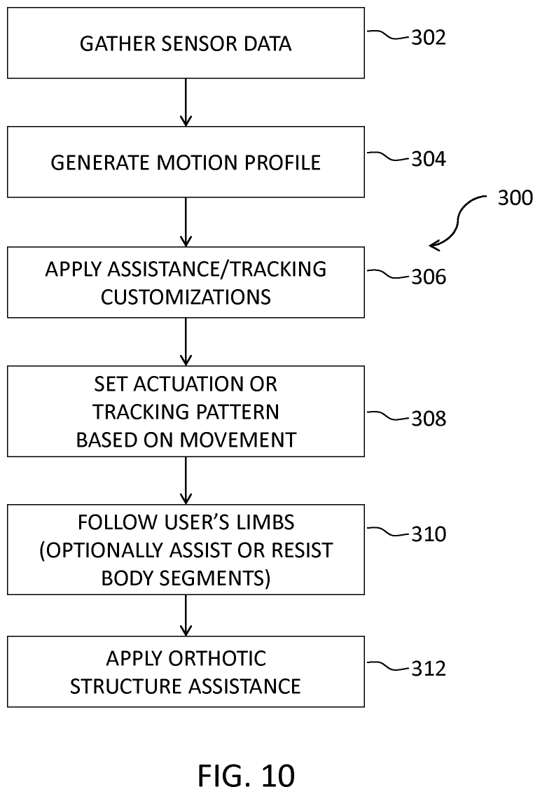

Referring now to FIG. 10, there is shown a flow diagram of the load distribution device control process 300 executed by the one or more processor 212 (see FIG. 9) in accordance with a first illustrative embodiment of the present disclosure. Steps of the process 300 are indicated by blocks 302 to 312.

The process 300 starts at block 302 where process 300 gathers mechanical and biomechanical information of the user acquired from the plurality of sensors 40, 45.

At block 304, the kinematics of the user's body segments are determined using a motion profiler and the gathered mechanical and biomechanical information from block 302.

At block 306, the type of tracking, assistance, and/or resistance offered to the user's limbs is chosen based on the type of application chosen for the system and on the user's customizations of the system settings.

Then, at block 308, the process 200 sets actuation or tracking patterns based on the motion detected by the motion profiler of block and on the user's customizations of block 306, and, at block 310, the process 300 directs the hip 22 and knee 23 actuators to apply either joint actuation to assist or resist the user's motion or provide tracking in a passive mode so as to follow and capture user's limbs kinematics data.

Finally, at block 312, the process 300 controls the load distribution device 10 so that is adapts to the user's natural body motion, allowing free movement unless assistance/resistance of the user's limbs is required.

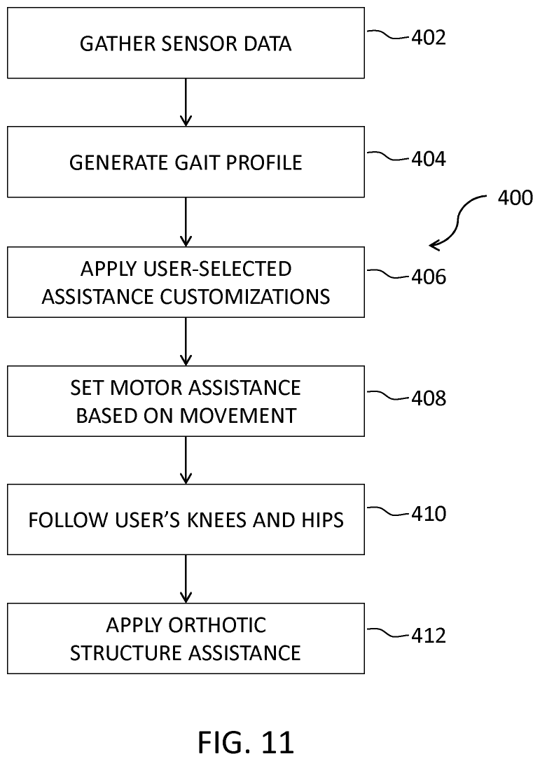

Referring to FIG. 11, there is shown a flow diagram of the load distribution device control process 400 executed by the one or more processor 212 (see FIG. 9) in accordance with a second illustrative embodiment of the present disclosure. Steps of the process 400 are indicated by blocks 402 to 412.

The process 400 starts at block 402 where process 400 gathers mechanical and biomechanical information of the user acquired from the plurality of sensors 40, 45.

At block 404, the kinematics of the user's body segments are determined using a gait profiler and the gathered mechanical and biomechanical information from block 402. The gait profiler may be for example, as disclosed in International Patent Application WO 2018/137016 A1 entitled “Gait Profiler System and Method” filed 25 Jan. 2017.

Then, at block 406, user customization of the magnitude and type of assistance offered by the load distribution device 10 are applied and, at block 408, the process 400 sets the level and timing of assistance offered to the wearer's hips and knees via the hip 22 and knee 23 actuators.

This allows, at block 410, the load distribution device 10 to follow the user's limbs using the detection of user movement pattern and intention provided by the gait profiler.

Finally, at block 412, the process 400 directs the hip 22 and knee 23 actuators to provide supplemental strength to the user to reduce the physiological demands of performing lower body activities. The load distribution device 10 also provides mechanical assistance to reduce the weight bearing burden on the user's joints.

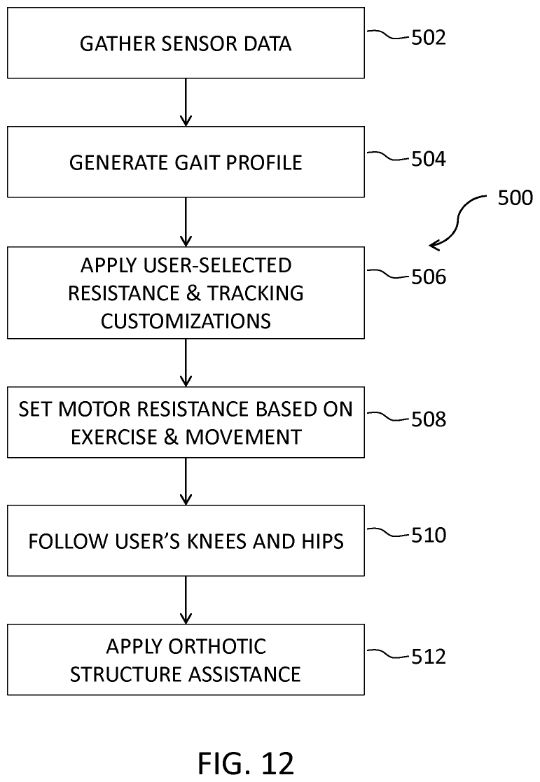

Referring to FIG. 12, there is shown a flow diagram of the load distribution device control process 500 executed by the one or more processor 212 (see FIG. 9) in accordance with a third illustrative embodiment of the present disclosure. Steps of the process 500 are indicated by blocks 502 to 512.

The process 500 starts at block 502 where process 500 gathers mechanical and biomechanical information of the user acquired from the plurality of sensors 40, 45.

At block 504, the kinematics of the user's body segments are determined using a gait profiler and the gathered mechanical and biomechanical information from block 502. The gait profiler may be for example, as disclosed in International Patent Application WO 2018/137016 A1 entitled “Gait Profiler System and Method” filed 25 Jan. 2017.

Then, at block 506, user customization of the magnitude and type of resistance as well as the type of exercise tracking and interactivity (e.g., tracking and signaling rest periods or repetitions) offered by the load distribution device 10 are applied, and, at block 508, the process 500 sets the level and timing of resistance offered to the wearer's hips and/or knees via the hip 22 and knee 23 actuators.

This allows, at block 508, the load distribution device 10 to follow the user's limbs while providing a set level and type of resistance.

Finally, at block 512, the process 500 directs the hip 22 and knee 23 actuators to provide resistance to oppose the motion of the user, allowing him or her to train strength in targeted ranges of motion and types of motion, for example constant joint speed, constant resistance force, targeted power, targeted pace, etc.

The selection of the type of tracking, assistance, and/or resistance offered to the user's limbs, magnitude and type of assistance provided, as well as the type of exercise tracking and interactivity may be performed using mechanical inputs (e.g., buttons) and/or digital inputs the located on the load distribution device 10 and/or via a software application on a peripheral device such as a remote control or a. smart phone.

It is to be understood that the various embodiments of the load distribution device disclosed therein may be provided with or without length adjustment capabilities and may be designed for specific user physiologies.

It is to be further understood that the load distribution device for transferring musculoskeletal stress from joints to body segments of the lower extremities of a user may be provided to one or both of the user's lower body extremities.

Although the present disclosure has been described by way of particular non-limiting illustrative embodiments and examples thereof, it should be noted that it will be apparent to persons skilled in the art that modifications may be applied to the present particular embodiment without departing from the scope of the present disclosure.

1. A load distribution device for improving the mobility of the center of mass of a user during complex motions, comprising:
a pelvic support belt configured to be positioned about a lower trunk of the user;
at least one thigh support element including two or more contact areas configured to be positioned in an agonist-antagonist configuration on a posterior part and an anterior part of a thigh of the user, the at least one thigh support element being rotationally connected to the pelvic support belt;
at least one hip joint actuator providing rotational motion of the at least thigh support element with respect to the pelvic support belt;
at least one shank support element including two or more contact areas configured to be positioned in an agonist-antagonist configuration on a posterior part and an anterior part of a shank of the user, the at least one shank support element being rotationally connected to the at least one thigh support element;
at least one knee joint actuator providing rotational motion of the at least one shank support element with respect to the at least one thigh support element;
a plurality of sensors positioned on the pelvic support belt, the at least one thigh support element, the hip joint actuator and the knee joint actuator, and at least one foot sensor configured to be positioned on a foot of the user, the plurality of sensors providing mechanical and biomechanical signals;
a control unit operatively connected to the plurality of sensors and at least one foot sensor for receiving the mechanical and biomechanical signals, the control unit having stored thereon executable instructions for processing and analyzing the mechanical and biomechanical signals and generating motions set-points of movements of the user; and
a power unit operatively connected to the at least one knee joint actuator, the at least one hip joint actuator and the control unit;
wherein the at least one knee joint actuator and the at least one hip joint actuator transfer musculoskeletal stress from joints to body segments of lower extremities of a user, and therefore improve stability of the joints and a range of motion of the body segments, by generating or dissipating biomechanical energy under directions of the control unit according to a computed level of energy corresponding to a musculoskeletal stress reduction at the joints of the lower extremities of the user necessary to compensate movements of the user, the generated or dissipated biomechanical energy being redistributed onto the lower trunk, the thigh and the shank of the user via the pelvic support belt, the at least one thigh support element and the at least one shank support element, respectively.

2. The load distribution device of claim 1, comprising two thigh support elements, two shank support elements, two hip joint actuators, two knee joint actuators and two feet sensors.

3. The load distribution device of claim 2, wherein each of the thigh support elements is rotationally connected to at least one of a shank support element via a knee pivot aligned with a center of rotation of a knee joint of the user and the pelvic support belt via a hip pivot aligned with a center of rotation of a hip joint of the user, and wherein at least one of the knee joint actuators and the hip joint actuators are located remotely from the center of rotation of an associated joint of the user, the at least one of the knee joint actuators and the hip joint actuators transmitting rotational motion to an associated joint of the user via an extension cable and a flexion cable.

4. The load distribution device of claim 3, wherein each of the knee joint actuators and each of the hip actuators are located in a location selected from the group consisting of medially on a respective side portion of the pelvic support belt, on a lower back portion of the pelvic support belt, on a respective front portion of the thigh of the user, on a respective back portion of the thigh of the user and on a respective portion of the thigh support element between a hip joint of the user and a knee joint of the user.

5. The load distribution device of claim 3, wherein the thigh support elements include a respective length adjustment mechanism.

6. The load distribution device of claim 5, wherein the length adjustment mechanism is selected from a group consisting of a slider mechanism and a screw mechanism.

7. The load distribution device of claim 5, wherein the extension cable and the flexion cable each include a tension mechanism.

8. The load distribution device of claim 3, further comprising a delocalization mechanism including a deportation structural link having at a first extremity an actuator support element configured to support one of the knee actuator and the hip actuator, and a second extremity having a pivot connection element for connecting a corresponding one of the knee pivot and the hip pivot.

9. The load distribution device of claim 8, wherein the actuator support element is configured to removably support the knee actuator the one of the knee actuator and the hip actuator.

10. The load distribution device of claim 3, wherein the extension cable and the flexion cable are formed by a single cable.

11-29. (canceled)

30. An orthotic device, comprising:
a proximal support element including at least one contact area configured to be secured to a proximal body portion of a user and distal support element including at least one contact area configured to be secured to a distal body portion of the user, the proximal support element and the distal support element being rotationally connected via a pivot aligned with a center of rotation of a corresponding joint of the user;
at least one actuator rotationally providing rotational motion of the distal support element with respect to the proximal support element, the at least one actuator being located remotely from the center of rotation of the corresponding joint of the user, the actuator transmitting rotational motion to the pivot via an extension cable and a flexion cable.

31. The orthotic device of claim 30, further comprising a delocalization mechanism including a deportation structural link having at a first extremity an actuator support element configured to support the actuator, and a second extremity having a pivot connection element for connecting to the pivot.

32. The orthotic device of claim 31, wherein the actuator support element is configured to removably support the actuator.

33. The orthotic device of claim 31, wherein the pivot is selected from a group consisting of a knee pivot, a hip pivot, an ankle pivot, an elbow pivot and a shoulder pivot.

34. (canceled)

35. The orthotic device of claim 31, wherein the extension cable and the flexion cable are formed by a single cable.
